(19) United States
(12) Patent Application Publication
(10) Pub. No.: US 2025/0178994 A1
(43) Pub. Date: Jun. 5, 2025

(54) CATALYST SYSTEM AND METHOD FOR PRODUCING HIGHER ALCOHOLS FROM METHANOL AND ETHANOL

(71) Applicant: FORSCHUNGSZENTRUM JÜLICH GMBH, Jülich (DE)

(72) Inventors: Johannes HÄUSLER, Moosburg an der Isar (DE); Joachim PASEL, Jülich (DE); Ralf PETERS, Würselen (DE); Friederike WOLTMANN, Bülkau (DE)

(73) Assignee: FORSCHUNGSZENTRUM JÜLICH GMBH, Jülich (DE)

(21) Appl. No.: 18/862,021

(22) PCT Filed: Apr. 21, 2023
(86) PCT No.: PCT/EP2023/060446

§ 371 Date: Oct. 31, 2024

(30) Foreign Application Priority Data
May 5, 2022 (DE) 10 2022 111 134.2

(51) Int. Cl. C07C 29/34; B01J 21/18; B01J 23/89; B01J 35/30; B01J 35/61; B01J 37/04; B01J 37/08; B01J 37/18

(52) U.S. Cl. CPC C07C 29/34; B01J 21/18; B01J 23/892; B01J 35/394; B01J 35/615; B01J 35/617; B01J 35/618; B01J 37/04; B01J 37/088; B01J 37/18

(57) ABSTRACT

The present invention relates to a process for the production of C3-C10 mono-alcohols from methanol and ethanol using a metal catalyst in the presence of a base, wherein a mixture comprising at least methanol and ethanol is reacted on a carbon-supported metal catalyst comprising at least one alloy of the metals nickel and platinum at a temperature of greater than or equal to 100° C. and less than or equal to 200° C., wherein the molar mass ratio of nickel to platinum is greater than or equal to 4 and less than or equal to 100,000. Furthermore, the present invention relates to a supported metal catalyst and the use of the catalyst for the preparation of C4-C8 mono-alcohols from methanol and ethanol.

The present invention relates to a process for the production of C3-C10 mono-alcohols from methanol and ethanol using a metal catalyst in the presence of a base, wherein a mixture comprising at least methanol and ethanol is reacted on a carbon-supported metal catalyst comprising at least one alloy of the metals nickel and platinum at a temperature of greater than or equal to 100° C. and less than or equal to 200° C., wherein the molar mass ratio of nickel to platinum is greater than or equal to 4 and less than or equal to 100,000. Furthermore, the present invention relates to a supported metal catalyst and the use of the catalyst for the production of C4-C8 mono-alcohols from methanol and ethanol.

Straight-chain or branched alcohols with medium C-numbers are important basic chemicals and reactants in the chemical industry, wherein the increased use as a fuel or fuel component in gasoline (e.g. i-butanol, 2-butanol, 1-butanol) and/or diesel fuels (1-octanol) makes an expansion of capacities appear likely. Based on the availability of relatively short-chain alcohols from biological sources, conversions that synthesize medium-chain alcohols from short-chain alcohols have regained importance in recent years. One well-known reaction is the Guerbet condensation, in which primary or secondary alcohols are converted to longer-chain alcohols at high temperatures and high pressure in the presence of alkali metal hydroxides or alkoxides via aldehydes and aldol products. For this basic reaction, there are a large number of different proposals for process control in the scientific and patent literature.

For example, U.S. Pat. No. 2,457,866A describes the synthesis of Guerbet alcohols starting from primary alcohols with at least 4 C atoms in the presence of a base and a hydrogenation catalyst at 120° C. to 300° C. with azeotropic distillation to remove water of reaction.

CN 108 043 403 A discloses a synthesis process of a CuO/MgO/M catalyst for the Guerbet reaction. M are oxides selected from the group Al₂O₃, ZrO₂, TiO₂, CeOx, SiO₂. The MgO:CuO:M ratio can be between 100:1-8:5-25. The synthesis involves the production of mesoporous MgO using cetylammonium bromide doped with Cu.

U.S. Pat. No. 2,050,788 A discloses a process for the synthesis of i-butanol by passing methanol and ethanol vapor over an MgO catalyst at normal and excess pressure and 200-400° C. An increased activity of the magnesium oxide can be achieved by Th, Pb, Ag, U, Cd, Sn, Cr, Mn, Zn, Fe, Ni, Co, Cu, their oxides or mixtures of the mentioned components. Different mixing ratios of methanol, ethanol and hydrogen in the reaction are described.

Such solutions known from the state of the art may offer further potential for improvement, particularly with regard to the process control at relatively low temperatures and with regard to a cost-effective and durable catalyst.

It is therefore the task of the present invention to at least partially overcome the disadvantages known from the prior art. In particular, it is the task of the present invention to provide a process which delivers high conversions and selectivity's at relatively low temperatures.

The problem is solved by the features of the respective independent claims, directed to the process according to the invention, the catalyst according to the invention and the use of the catalyst according to the invention for the synthesis of longer-chain alcohols. Preferred embodiments of the invention are given in the independent claims, in the description or in the FIGURES, wherein further features described or shown in the dependent claims or in the description or in the FIGURES may individually or in any combination constitute an object of the invention, as long as the context does not clearly indicate the contrary.

According to the invention, a process for the preparation of C3-C10 mono-alcohols from methanol and ethanol by means of a metal catalyst in the presence of a base, wherein a mixture comprising at least methanol and ethanol is reacted on a carbon-supported metal catalyst comprising at least one alloy of the metals nickel and platinum at a temperature of greater than or equal to 100° C. and less than or equal to 200° C., wherein the molar mass ratio of nickel to platinum, expressed as molar amount of nickel divided by molar amount of platinum, is greater than or equal to 4 and less than or equal to 100.000.

Surprisingly, it was found that the reaction according to the invention is characterized by high conversions and selectivity's even at low catalyst concentrations. Without being bound by theory, the temperature range in particular appears to be suitable for preventing the formation of unwanted by-products at all or only in small quantities. The process can be operated with widely varying alcohol reactants and ratios and is characterized by long catalyst lifetimes and high space-time yields. Another advantage is that the additional use of hydrogen can be completely dispensed with. It was also found that, in contrast to mixed metal oxide catalyst systems, which are complex to produce and require high pressures of up to 100 bar during conversion, there is no need for an increased pressure range during conversion. In contrast to the aforementioned prior art, no expensive rare earths, such as lanthanide oxide, are required. The carbon-rich carrier material is sufficiently mechanically stable, and this design of the catalyst means that the catalyst has a good product formation rate even in the presence of water and produces higher alcohols with a high degree of efficiency.

The process according to the invention is a process for the production of C3-C10 mono-alcohols from methanol and ethanol. In the process according to the invention, mixtures of short-chain mono-alcohols with one or two carbon atoms are thus converted to mono-alcohols with more than two carbon atoms. The medium-chain alcohols can be straight-chain or branched. The starting material mixture used for the synthesis can have varying proportions of ethanol and methanol. Preferably, the molar ratio of methanol to ethanol can be between 20:1 and 0.5:1, more preferably between 14:1 and 1:1. In principle, it is also possible to convert pure ethanol or pure higher alcohols or mixtures of at least two components of the higher alcohols by means of the process according to the invention. As a function of the mixture used, the reaction temperature and, for example, the residence time, a mixture of alcohols with different C numbers is usually obtained, the isolated pure alcohols being obtainable by means of conventional separation operations. Products available include propanol, n/i-butanol and decanol. Alcohols with even longer C-chains can also be formed, but the selectivity and yield of these even higher alcohols is rather unsatisfactory.

The mixture for the reaction comprises at least methanol and ethanol and the mixture is reacted on a carbon-supported metal catalyst. The reaction of the short-chain alcohols takes place on a metal catalyst, which is not present as such but is bound to a carrier material. The actual metal catalyst or the metal alloy is preferably chemically or physically bound on the surface or in the carrier material. The carrier material can be used in the form of powders or molded bodies. A suitable shaped body geometry is, for example, spherical or ellipsoidal. The carriers can, for example, be monodisperse or polydisperse particles. Suitable carbon-containing carrier materials include powdered activated carbon, granulated activated carbon, ordered and disordered mesoporous carbon, carbon black or single-walled and multi-walled carbon nanotubes or mixtures of at least two components thereof.

The reaction takes place in the presence of a base. The bases known to the skilled person can be used for the catalytic reaction. For example, alkali or alkaline earth hydroxides can be used.

The metal catalyst comprises an alloy of the metals nickel and platinum. The metal catalyst used therefore does not consist of individual metals, but of an alloy comprising at least the metals nickel and platinum. The alloy of these two metals can be produced from the individual metals in different compositions using processes known to the skilled person, for example by thermal treatment. Preferably, the alloy can also consist of nickel and platinum.

The temperature in the reaction is greater than or equal to 100° C. and less than or equal to 200° C. The reaction according to the invention can therefore be carried out within a relatively small temperature window and at relatively low temperatures compared to the processes known from the prior art. In particular, this temperature selection can result in a small amount of unwanted by-products and a long service life of the catalyst.

The molar mass ratio of nickel to platinum in the catalyst, expressed as the molar amount of nickel divided by the molar amount of platinum, is greater than or equal to 4 and less than or equal to 100,000. Within these ratios in the composition of the alloy, very efficient catalysts can be produced, which are characterized by high conversion and good selectivity's. The catalysts are mechanically stable and show only a slight loss of activity even after long periods of standing under reaction conditions. Preferably, the molar ratio can also be greater than or equal to 10 and less than or equal to 75,000, furthermore preferably also greater than or equal to 15 and less than or equal to 50,000. Preferably, the alloy of the metal catalyst can consist of the components nickel and platinum.

In a preferred embodiment of the process, the molar ratio of nickel to platinum can be greater than or equal to 20 and less than or equal to 100. In the reaction according to the invention, it has been found to be particularly advantageous that the ratio of the metals used in the alloy is in the range specified above. Long catalyst lifetimes can be achieved with a high selectivity to the longer-chain alcohols with high space-time yields. This composition also has the advantage that the proportion of relatively expensive platinum in the catalyst is very small. This can reduce the process costs of the conversion.

In a further preferred embodiment of the process, the carrier material of the carbon-supported metal catalyst can comprise greater than or equal to 75% by weight of amorphous carbon. It has been found to be very advantageous for the conversion that the carrier material has a very large proportion of carbon. This can have a positive effect on the efficiency of the conversion. In particular, the carrier material of the catalyst can consist of carbon. Surprisingly, it has also been shown that the use of an amorphous carbon, without close arrangement of the individual carbons to each other, can contribute to improved conversions. A carbon is an amorphous carbon if no crystalline phases can be detected in the carbon phase in an XRD measurement with Cu-alpha wavelength under Bragg-Brentano geometry. Crystalline phases for the different carbon materials result, for example, from significant reflections of graphite (26.6°, 42.3°, 44.5°, 50.7°, 54.7°) , diamond) (44.0°, C60 (21.0°, 22.0°, 27.7°, 28.4°, 31.2°, 33.1°) and are known to the skilled person.

Within a further preferred aspect of the process, the support material of the carbon-supported metal catalyst may have an internal surface area, determined by static physisorption of N₂, of greater than or equal to 500 m²/g and less than or equal to 1300 m²/g. For the efficiency of the reaction and for a long service life of the catalyst under reaction conditions, the above-mentioned range of surface areas has proven to be particularly suitable. The surface area of the support can be determined by means of static physisorption of N₂ in a range of p/p₀=0.01 to 0.99 at 77.15K on a Gemini VII from Micromeritics. It is advisable to degas the sample for 1 h at 150° C. and 0.007 mbar before the measurement. Higher surface areas can be detrimental to the service life of the catalyst. Smaller surface areas can lead to insufficient conversion of the feed alcohols.

According to a preferred characteristic of the process, the temperature can be greater than or equal to 130° C. and less than or equal to 170° C. Within this very limited temperature range, a large number of different methanol/ethanol reactant mixtures can be converted with high conversions and very good selectivity. In addition to the flexibility in the application ratio, the catalyst shows only a slight decrease or deactivation over the reaction time, resulting in very constant space-time yields over the conversion.

In a further preferred embodiment of the process, the mass fraction of the metal alloy in the total mass of the catalyst can be greater than or equal to 3% by weight and less than or equal to 15% by weight. Within this mass ratio between active catalyst metal alloy and carbon-containing carrier material, mechanically very stable and extremely efficient catalysts can be provided. The catalysts are characterized by high conversion rates and very good selectivity and have a long service life under reaction conditions. Furthermore, the mass ratio between active catalyst metal alloy and carbon-containing carrier material can be greater than or equal to 2.0 wt. % and less than or equal to 14.0 wt. %, furthermore preferably greater than or equal to 3.0 wt. % and less than or equal to 12.5 wt. %.

Within a preferred aspect of the process, the carbon supported metal catalyst can have a size distribution with a number averaged D05 quantile determined via TEM of greater than or equal to 0.5 nm and less than or equal to 5 nm. Surprisingly, it has been shown that particulate supported catalysts with the above size specification contribute to particularly efficient conversions of a variety of different methanol/ethanol mixtures. The supported catalysts show high conversions even at relatively low temperatures and are characterized by long service lives. Preferably, the D05 quantile can be greater than or equal to 1.0 nm and less than or equal to 4.5 nm, further preferably greater than or equal to 1.5 nm and less than or equal to 4.0 nm. Further preferably, the D09 quantile can be greater than or equal to 5.0 nm and less than or equal to 12.0 nm, further preferably greater than or equal to 6.0 nm and less than or equal to 10.0 nm.

In a further embodiment of the process, the catalyst concentration may preferably be greater than or equal to 0.7 g/L and less than or equal to 14.5 g/L, preferably greater than or equal to 1.4 g/L and less than or equal to 10.7 g/L, further preferably greater than or equal to 1.42 g/L and less than or equal to 7.5 g/L.

Further according to the invention is a metal catalyst, wherein the metal catalyst comprises at least:
a) a carbonaceous carrier material in a proportion by weight greater than or equal to 80% and less than or equal to 95%;
b) a metal alloy comprising nickel and platinum, wherein the molar proportion of nickel in the alloy is greater than or equal to 80% and less than or equal to 99.99% and the molar proportion of platinum in the alloy is greater than or equal to 0.01% and less than or equal to 20%; wherein the carbon is amorphous.

This catalyst is characterized by particularly favourable mechanical properties and a high conversion efficiency, especially for the conversion of ethanol and methanol to longer-chain mono-alcohols. For further advantages of the catalyst, particular reference is made to the advantages of the catalyst in the process according to the invention.

Within a further preferred embodiment of the metal catalyst, the catalyst can have an internal surface area, determined by means of static physisorption of N₂, of greater than or equal to 500 m2/g and less than or equal to 1300 m²/g. The above surface area range has proven to be particularly suitable for a well-balanced catalyst with high conversion efficiency and high mechanical strength. The catalyst is characterized by particularly low mechanical abrasion and low catalyst losses during conversion.

Further according to the invention is the use of a metal catalyst according to the invention for the production of C4-C8 mono-alcohols from methanol and ethanol. The catalyst claimed according to the invention can be used particularly advantageously in processes in which alcohols with lower C numbers are converted to alcohols with higher C numbers. For the advantages of using the catalyst, explicit reference is made to the advantages of the process according to the invention. In principle, the reaction can also be carried out with pure ethanol or pure higher alcohols (>C2) or mixtures of the higher alcohols on the catalyst according to the invention using the process according to the invention.

In a preferred embodiment of use, the process can be used to synthesize i-butanol. For a high selectivity of i-butanol in the process according to the invention, a concentration of 0.1 to 2.5 mol/L, but preferably 0.5 to 1.7 mol/L ethanol in a methanolic solution can preferably be maintained. At higher ethanol concentrations, more products can be obtained which unintentionally contain a mixture of higher alcohols.

### EXAMPLES

### I. Preparation of Supported Catalysts According to the Invention

### I.a Impregnation of a Carrier

To prepare a precursor solution, precursor compounds of nickel and platinum are dissolved in deionized water. The nickel salts in the form of chlorides, nitrates, sulphates and acetates are suitable for nickel. For platinum, the free hexachloroplatin (IV) acid and its sodium and potassium salts, as well as [Pt(NH₃)₄](NO₃)₂ are suitable. The volume of the solution is diluted so that it corresponds to the water absorption capacity of the carbon-rich carrier material. Approx. 5 g of the carbon-rich carrier material is mixed with the precursor solution in a vessel and mechanically homogenized. The catalyst precursor is then pre-dried at an elevated temperature of 30-100° C., under reduced pressure of 1000-10 mbar for 0.1-4 h with continuous mechanical mixing. In an inert gas stream, for example nitrogen, calcination takes place at a mass flow rate of 0.1-2 g/min and a duration of 1-7 h at a temperature of 400-700° C. and then the temperature is reduced to 100-300° C. At this temperature, a maximum of 50 vol % H₂ is added to the inert gas stream for 0.5-2 h and the catalyst precursor is treated. After this treatment, the metals are alloyed and the catalyst precursor is referred to as a catalyst.

### I.b Impregnation of a Carbonaceous Carrier

An alternative way to produce the catalysts is by precipitating nickel and subsequently impregnating and alloying Pt onto activated carbon. For this purpose, a 1-0.05 mol/L aqueous solution is prepared with suitable nickel compounds, in which the carbon-rich carrier material is suspended. The compounds of chlorides, nitrates, sulphates and acetates of nickel are suitable for this purpose. The concentration of the solution, the solution volume and the quantity of the carbon-rich carrier material are determined in such a way that the desired loading is obtained with complete precipitation. The precipitation is carried out with an aqueous solution of a suitable base such as ammonia, KOH, K₂O, BaOH, Ca(OH)₂, CaO, Na₂O, the sodium, potassium or ammonium salts of hydrogen carbonate and carbonate or NaOH. The solution contains a concentration of OH ions of 0.8-0.02 mol/L and is added at 0.1-5 mL/min with continuous mixing of the suspension. During precipitation, the temperature of the suspension is maintained at 20-80° C. The end of the precipitation is monitored using a pH electrode and determined at a maximum of pH=12. The suspension is then aged at room temperature with constant stirring for 0.5-4 hours and filtered off. The filter cake is washed with deionized water until a conductivity<400 μS/cm of the filtrate is achieved. The catalyst precursor is then pre-dried at an elevated temperature of 30-100° C. under reduced pressure of 1000-10 mbar for 0.1-4 h with continuous mechanical mixing. The dried catalyst precursor is impregnated with an aqueous precursor solution containing suitable platinum compounds in order to achieve the desired Ni:Pt ratio. The free hexachloroplatin (IV) acid and its sodium and potassium salts, as well as [Pt(NH₃)₄](NO₃)₂ are suitable for this purpose. The catalyst precursor is then pre-dried at an elevated temperature of 30-100° C. under reduced pressure of 1000-10 mbar for 0.1-4 h with continuous mechanical mixing. Under an inert gas stream, calcination takes place at a mass flow rate of 0.1-2 g/min and a duration of 1-7 h at a temperature of 400-700° C. and then the temperature is reduced to 100-300° C. At this temperature, a maximum of 50 vol % H₂ is added to the inert gas stream for 0.5-2 h and the catalyst precursor is treated. After this treatment, the catalyst precursor is referred to as a catalyst with an alloy of nickel and platinum.

### I.c Testing the Catalyst Activity

The chemical reaction for testing the catalysts is carried out in an autoclave reactor. The autoclave walls and internals are made of Inconell 600 and the autoclave has an impeller stirrer. The autoclave is loaded and sealed with, for example, 0.05-5 g of catalyst of the particle size fraction d<500 μm, but preferably d<75 μm. The atmosphere is exchanged with one or more of the inert gases helium, nitrogen or argon. 70 mL of the reaction solution is added via a septum. The reaction solution consists of a mixture of methanol (pure, Merck KGAA) and ethanol (pure, Merck KGaA) and deionized water in different concentration ratios, as well as NaOH (ACS, >97%) with a concentration of 0.2 mol/L-2 mol/L, but preferably 0.4 mol/L to 0.8 mol/L and n-decane as internal standard. The reaction is carried out at 150 to 200° C. for 4 h with a stirrer speed of 1000 rpm and autogenous pressure under a nitrogen atmosphere. The time at which the target temperature is reached defines the start of the reaction time. At 30 min intervals, 3 μL of the reaction solution is quantitatively analyzed by headspace method (full evaporation) on GC-MS (Agilent 8890, 122-7032 UI DB-Wax column) with an Agilent 5977GC/MSD mass spectrometer.

### II Examples

### II.1 Example 1

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 0.6 mol/L ethanol (pure, Merck KGAA), 0.45 mol/L NaOH (>97%, Acros Organics) 0.015 mol/L n-decane (99%, Acros Organics) as internal standard. The reaction is carried out at 120° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 1.

### II.2 Example 2

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 0.6 mol/L ethanol (pure, Merck KGAA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At intervals of 60 min, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 1.

### II.3 Example 3

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 0.6 mol/L ethanol (pure, Merck KGAA), 0.45 mol/L NaOH (>97%, Acros Organics) 0.015 mol/L n-decane (99%, Acros Organics) as internal standard. The reaction is carried out at 180° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 1.

### II.4 Example 4

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 0.6 mol/L ethanol (pure, Merck KGAA), 0.45 mol/L NaOH (>97%, Acros Organics) 0.015 mol/L n-decane (99%, Acros Organics) as internal standard. The reaction is carried out at 200° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 1.

### II.5 Example 5

1.2264 g Ni(NO₃)₂-6H₂O (>98%, Thermo Scientific) and 0.0049 g [Pt(NH₃)₄](NO₃)₂ (Alfa Aesar) are dissolved in 2.350 g deionized water. 4.8766 g activated carbon (Merck KgGaA) are added to the precursor solution in a tightly sealable vessel and homogenized. The catalyst precursor is then pre-dried at 40° C., 6 rp,m and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the prepared catalyst 5% Ni₉₉ Pt₁/C (grain size d<75 μm) is weighed into the autoclave reactor and the autoclave is sealed. The atmosphere is exchanged with nitrogen. Add 70 mL of the reaction solution via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 1.

### II.6 Example 6

0.25 g of the catalyst 5% Ni₉₉ Pt₁/C (particle size d<75 μm) produced in example 5 is weighed into the autoclave reactor and the autoclave is sealed. The atmosphere is exchanged with nitrogen. Add 70 mL of the reaction solution via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 1.

TABLE 1

| No. | Catalyst | Temperature | X(EtOH)/% | S(i-butanol)/% |
|---|---|---|---|---|
| 1 | 5% Pt/C | 120° C. | 3.89 | 3.68 |
| 2 | 5% Pt/C | 150° C. | 2.76 | 88.93 |
| 3 | 5% Pt/C | 180° C. | 3.44 | 97.70 |
| 4 | 5% Pt/C | 200° C. | 4.85 | 96.45 |
| 5 | 5% Ni₉₉Pt₁/C | 150° C. | 1.67 | 99.90 |
| 6 | 5% Ni₉₉Pt₁/C | 165° C. | 4.91 | 99.90 |

The examples 1-6 show that the platinum catalyst (example 1-4) has a strong tendency to deactivate, particularly at temperatures above 150° C. This becomes clear in Table 1 from the fact that the concentration of i-butanol rises sharply at the beginning but increases less sharply as the reaction progresses. At 180° C. (No. 3), the catalyst is already inactive for the formation of further i-butanol after two hours. A comparison of No. 2 and No. 5 shows that the 5% Ni₉₉Pt₁/C catalyst produces a lower conversion of ethanol. At the same time, the selectivity to i-butanol is increased by 8%. The increase in the i-butanol concentration is linear at 150° C. for the 5% Ni₉₉ Pt₁/C catalyst, while the Pt/C catalyst already shows a slight decrease in the increase in concentration. At No. 6, higher conversions of ethanol are already achieved than with the 5% Pt/C catalyst at 200° C. (No. 4). This clearly shows that the 5% Ni₉₉Pt₁/C catalyst has a higher stability and therefore achieves higher conversions in the same period of time.

### II.7 Example 7

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 1.2 mol/L ethanol (pure, Merck KGAA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.8 Example 8

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 1.60 mol/L ethanol (pure, Merck KGAA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.9 Example 9

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 1.96 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.10 Example 10

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 3.02 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.11 Example 11

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 3.84 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At intervals of 60 min, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.12 Example 12

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 5.95 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 60 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.13 Example 13

0.25 g of a classified (grain size d<75 μm) commercial platinum catalyst (5% Pt/C, reduced, Alfa Aesar) is weighed into the autoclave reactor and the autoclave is sealed. The atmosphere is exchanged with nitrogen. Add 70 mL of the reaction solution via a septum. The methanolic reaction solution contains 10.11 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At intervals of 60 min, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 2.

### II.14 Example 14

1.0544 g Ni(NO₃)₂-6H₂O (>98%, Thermo Scientific) and 0.0739 g [Pt(NH₃)₄] (NO₃₂ (Alfa Aesar) are dissolved in 2.0106 g deionized water. 4.5000 g activated carbon (Merck KgGaA) are mixed with the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the prepared catalyst 5% Ni₉₅Pt₅/C (grain size d<75 μm) is weighed into the autoclave reactor and the autoclave is sealed. The atmosphere is exchanged with nitrogen. Add 70 mL of the reaction solution via a septum. The methanolic reaction solution contains 2.0 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 2.

### II.15 Example 15

0.25 g of the catalyst 5% Ni₉₅Pt₅/C (particle size d<75 μm) produced in example 14 is weighed into the autoclave reactor and the autoclave is sealed. The atmosphere is exchanged with nitrogen. Add 70 mL of the reaction solution via a septum. The reaction solution contains ethanol (pure, Merck KGaA) as solvent, 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 2.

TABLE 2

| | | | | Selectivity/% | | | | |
|---|---|---|---|---|---|---|---|---|
| | | c₀ (EtOH) | X(EtOH) | | Ethyl | | Ethyl | Ethyl |
| No. | MeOH:EtOH | mol/L | % | Acetaldehyde | formate | Butanal | acetate | propionate |
| 7 | 20:1 | 1.20 | 1.17 | — | — | — | — | — |
| 8 | 15:1 | 1.60 | 1.31 | 21.92 | — | — | — | — |
| 9 | 10:1 | 1.96 | 0.80 | 9.55 | — | — | 0.32 | — |
| 10 | 7:1 | 3.02 | 0.90 | 0.38 | — | — | — | — |
| 11 | 5:1 | 3.84 | 0.48 | 0.10 | — | — | — | 17.71 |
| 12 | 2:1 | 5.95 | 0.22 | 0.13 | — | — | — | — |
| 13 | 1:1 | 10.11 | 0.17 | 3.55 | 0.41 | 0.03 | 0.18 | — |
| 14 | 9:1 | 2.0 | 1.64 | — | — | — | — | — |
| 15 | EtOH | 17.04 | 6.51 | 2.65 | — | 24.75 | — | — |

| | Selectivity/% | | | | | | | |
|---|---|---|---|---|---|---|---|---|
| | 2-Methyl- | | 2-Methyl- | 2-Ethyl- | 2-Methyl- | | 2-Ethyl- | |
| No. | 1-propanol | 1-Butanol | 1-butanol | 1-butanol | 1-pentanol | 1-Hexanol | 1-hexanol | 1-octanol |
| 7 | 52.91 | — | 25.03 | — | 7.52 | — | — | — |
| 8 | 44.59 | — | 20.20 | — | 6.00 | — | — | — |
| 9 | 28.85 | 12.96 | 27.59 | 7.30 | 6.49 | — | — | — |
| 10 | 25.56 | 18.56 | 30.77 | 5.76 | 6.42 | 5.15 | — | — |
| 11 | 14.11 | 36.94 | 4.26 | 9.43 | 6.43 | — | — | — |
| 12 | 7.22 | 31.04 | 28.49 | 9.26 | 11.18 | 7.31 | — | — |
| 13 | 1.00 | 44.87 | 12.26 | 9.18 | 11.61 | 9.67 | 0.11 | 3.40 |
| 14 | 49.41 | 3.55 | 28.98 | — | 2.61 | 14.22 | — | — |
| 15 | — | 47.04 | 0.02 | 11.66 | — | 2.52 | 6.04 | 5.31 |

Embodiments 7 to 15 show the dependence of the product distribution on the initial ethanol concentration. At low ethanol concentrations (a high methanol to ethanol ratio), the formation of i-butanol and 2-methyl-1-butanol predominates. In examples 7 and 8, for example, the selectivity to i-butanol is 52.91% and to 2-methyl-1-butanol 25.03%. i-Butanol is formed by double methylation of ethanol with 1-propanol as an intermediate. 2-Methyl-1-butanol is formed from the crossed aldol addition of 1-propanol and ethanol or from the subsequent methylation of 1-butanol, which is formed by the homo-aldol condensation and rehydration of ethanol.

With increasing concentrations of ethanol, the rate of homo-aldol condensation and rehydration of ethanol increases. Therefore, the selectivity to i-butanol decreases rapidly with slightly increasing ethanol concentrations from 52.91% (No. 7) to 28.85% (No. 10) while the ethanol concentration has only increased by 0.76 mol/L (+63%). Therefore, 1-butanol and its homo-aldol condensation product 2-ethyl-1-butanol are already formed in example 9. Alternatively, 2-ethyl-1-butanol could also be formed from the heterocondensation of ethanol with 1-butanol. This trend is further illustrated in the following examples. So that finally at a concentration of 10 mol/L ethanol (MetOH:EthOH=1:1) 1-octanol can already be formed.

For examples 14 and 15, the 5% Ni₉₅Pt₅/C catalyst was used at 165° C. No. 14 is comparable to No. 10 in terms of ethanol concentration. It can be seen that the 5% Ni₉₅Pt₅/C catalyst has a higher selectivity to i-butanol than the Pt/C catalyst at higher ethanol concentrations. At the same time, 1-hexanol with a selectivity of 14.22% is already formed at concentrations of 2 mol/L ethanol. 1-Hexanol can only be formed by the nucleophilic addition of ethanol (or acetaldehyde) to 1-butanol (or butanal). This is remarkable because normally the higher substituted alcohol is the better nucleophile and therefore the formation of 2-ethyl-1-butanol should be preferred. The 5% Ni₉₅Pt₅/C catalyst thus exhibits a higher selectivity to linear coupling products than the commercially available 5% Pt/C catalyst.

### II.16 Example 16

1.2388 g Ni(NO₃)₂-6H₂O is dissolved in 42.59 g deionized water and suspended in 4.75 g activated carbon. Using a Metrohm Titrando 808, precipitate quantitatively with sodium hydroxide solution (0.1 mol/L, ACS, >97%, addition rate=0.5 mL/min) under stirring at room temperature, age for one hour and filter off. The filter cake is washed with deionized water until a conductivity<400 μS/cm of the filtrate is reached. The catalyst precursor is then pre-dried at 40° C., 6 rpm and 40 mbar in a rotary evaporator. 0.0049 g [Pt(NH₃)₄](NO₃)₂ is dissolved in 0.8997 g deionized water and impregnated onto the nickel-loaded catalyst precursor. The impregnated catalyst precursor is pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the prepared catalyst 5% Ni₉₉ Pt₁/C (grain size d<75 μm) is weighed into the autoclave reactor and the autoclave is sealed. The atmosphere is exchanged with nitrogen. Add 70 mL of the reaction solution via a septum. The methanolic reaction solution contains 1.6 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.17 Example 17

0.25 g of the catalyst 5% Ni₉₉ Pt₁/C (particle size d<75 μm) produced according to example 5 is weighed into the autoclave reactor without prior treatment with hydrogen and the autoclave is sealed. The atmosphere is exchanged with nitrogen. 70 mL of the reaction solution is added via a septum. The methanolic reaction solution contains 1.6 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 3.

### II.18 Example 18

0.25 g of the catalyst 5% Ni₉₉ Pt₁/C (particle size d<75 μm) produced according to example 5 is weighed into the autoclave reactor after the previous treatment with hydrogen and the autoclave is sealed. The atmosphere is exchanged with nitrogen. 70 mL of the reaction solution is added via a septum. The methanolic reaction solution contains 1.6 mol/L ethanol (pure, Merck KGaA), 0.45 mol/L NaOH (>97%, Acros Organics) 0.015 mol/L n-decane (99%, Acros Organics) as internal standard. The reaction is carried out at 150° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 3.

### II.19 Example 19

0.25 g of the catalyst 5% Ni₉₉ Pt₁/C (particle size d<75 μm) produced according to example 5 is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.20 Example 20

2.3972 g Ni(NO₃)₂-6H₂O (>98%, Thermo Scientific) and 0.0322 g [Pt(NH₃)₄](NO₃)₂ (Alfa Aesar) are dissolved in 2.0257 g deionized water. 4.4977 g activated carbon (Merck KgGaA) is added to the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the produced catalyst 10% Ni₉₉ Pt₁/C (grain size d<75 μm) is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.21 Example 21

4.7944 g Ni(NO₃)₂-6H₂O (>98%, Thermo Scientific) and 0.0645 g [Pt(NH₃)₄](NO₃)₂ (Alfa Aesar) are dissolved in 1.8006 g deionized water. 4.0066 g activated carbon (Merck KgGaA) is mixed with the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the produced catalyst 20% Ni₉₉ Pt₁/C (grain size d<75 μm) is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.22 Example 22

7.1916 g Ni(NO₃)₂-6H₂O (>98%, Thermo Scientific) and 0.0967 g [Pt(NH₃)₄](NO₃)₂ (Alfa Aesar) are dissolved in 1.5756 g deionized water. 3.5007 g activated carbon (Merck KgGaA) is added to the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the produced catalyst 30% Ni₉₉ Pt₁/C (grain size d<75 μm) is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.23 Example 23

2.1088 g Ni(NO₃)₂-6H₂O (>98%, Thermo Scientific) and 0.1478 g [Pt(NH₃)₄] (NO₃)₂ (Alfa Aesar) are dissolved in 2.0257 g deionized water. 4.4948 g activated carbon (Merck KgGaA) are added to the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the produced catalyst 10% Ni₉₅Pt₅/C (grain size d<75 μm) is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.24 Example 24

4.2126 g Ni(NO₃)₂-6H₂O (>98%, Thermo Scientific) and 0.3008 g [Pt(NH₃)₄](NO₃)₂ (Alfa Aesar) are dissolved in 1.8168 g deionized water. 4.0049 g activated carbon (Merck KgGaA) are added to the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then 20 the temperature is reduced to 250° C. At this temperature, 2 vol % H₂ is added for 60 min and the catalyst precursor is treated. After this treatment, the catalyst precursor is referred to as the catalyst.

0.25 g of the produced catalyst 20% Ni₉₅Pt₅/C (grain size d<75 μm) is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

### II.25 Example 25

6.3339 g Ni(NO₃)₂-6H₂O (>98%, Thermo Scientific) and 0.4432 g [Pt(NH₃)₄](NO₃)₂ (Alfa Aesar) are dissolved in 1.5710 g deionized water. 3.5029 g activated carbon (Merck KgGaA) are added to the precursor solution in a tightly sealable vessel and homogenized. The catalyst precursor is then pre-dried at 40° C., 6 rpm and 40 mbar in a rotary evaporator. In the tube furnace, calcination is carried out in a nitrogen stream of 0.5 g/min for 4 h at 500° C. and then the temperature is reduced to 250° C. At this temperature, 2 vol % H₂ is added for 60 min and the catalyst precursor is treated. After this treatment, the catalyst precursor is referred to as the catalyst.

0.25 g of the produced catalyst 30% Ni₉₅Pt₅/C (grain size d<75 μm) is weighed into the autoclave reactor after the previous treatment with hydrogen and the autoclave is sealed. The atmosphere is exchanged with nitrogen. 70 mL of the reaction solution is added via a septum. The methanolic reaction solution contains 0.6 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) with an Agilent 5977 GC/MSD mass spectrometer. The results are listed in Table 3.

### II.26 Example 26 (Blind Test)

For a blind test, the autoclave reactor is closed without a catalyst. The atmosphere is exchanged with nitrogen. Add 70 mL of the reaction solution via a septum. The methanolic reaction solution contains 0.6 mol/L ethanol (pure, Merck KGAA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 3.

TABLE 3

| | | c(EtOH) | T(reaction) | X(EtOH) |
|---|---|---|---|---|
| No. | Catalyst | mol/L | ° C. | % |
| 16 | 5% Ni₉₉Pt₁/Cₖₒₙₛ | 1.60 | 150 | 1.29 |
| 17 | 5% Ni₉₉Pt₁/C | 1.60 | 150 | 0.60 |
| 18 | 5% Ni₉₉Pt₁/C_{red} | 1.60 | 150 | 0.97 |
| 19 | 5% Ni₉₉Pt₁/C | 0.60 | 165 | 6.13 |
| 20 | 10% Ni₉₉Pt₁/C | 0.60 | 165 | 7.65 |
| 21 | 20% Ni₉₉Pt₁/C | 0.60 | 165 | 9.13 |
| 22 | 30% Ni₉₉Pt₁/C | 0.60 | 165 | 13.45 |
| 23 | 10% Ni₉₅Pt₅/C | 0.60 | 165 | 18.67 |
| 24 | 20% Ni₉₅Pt₅/C | 0.60 | 165 | 11.62 |
| 25 | 30% Ni₉₅Pt₅/C | 0.60 | 165 | 8.63 |
| 26 | Blind test | 0.60 | 165 | 0.47 |

Selectivity in %

| | | | 1- | 2-Methyl- | 1- | 2-Methyl- | 2-Ethyl- | 2-Methyl- |
|---|---|---|---|---|---|---|---|---|
| No. | Acetaldehyde | Acetone | propanol | 1-propanol | Butanol | 1-butanol | 1-butanol | 1-pentanol |
| 16 | — | 1.11 | 1.89 | 85.88 | — | 11.12 | — | — |
| 17 | — | — | 4.37 | 52.95 | 5.90 | 21.74 | 15.03 | — |
| 18 | — | 2.95 | 3.10 | 77.59 | — | 16.36 | — | — |
| 19 | — | 1.66 | 1.14 | 97.20 | — | — | — | — |
| 20 | 0.30 | 1.25 | 1.17 | 93.05 | — | 4.22 | — | — |
| 21 | — | 0.67 | 1.63 | 90.66 | — | 5.18 | — | 1.86 |
| 22 | 0.44 | 1.24 | 1.34 | 92.94 | — | 4.05 | — | — |
| 23 | — | — | 2.23 | 90.73 | — | 5.46 | — | 1.59 |
| 24 | — | 0.55 | 2.12 | 91.40 | — | 5.93 | — | — |
| 25 | — | — | 3.48 | 84.84 | — | 8.54 | — | 3.14 |
| 26 | — | — | — | 27.88 | — | 72.12 | — | — |

The embodiment examples 16 to 18 show the influence of the different pre-treatment methods on the catalyst activity under otherwise identical reaction conditions. Example 16 shows a catalyst obtained by precipitation and subsequent impregnation according to method 2.

Compared to example 18, 85% selectivity to i-butanol and 1.3% ethanol conversion are obtained. In contrast, the catalyst 5% Ni₉₉ Pt₁/C_{red} from embodiment example 18 produces only 0.97% ethanol conversion with a selectivity of 77.59% to i-butanol. A comparison of No. 17 and No. 18 clearly shows that the reduction in the hydrogen stream has an advantageous effect for the catalyst in embodiment example 18. Thus, the ethanol conversion can be increased by 0.37% and the selectivity to i-butanol by 24.64% compared to the catalyst not treated in the hydrogen stream (No. 17).

Increasing the reaction temperature results in an increase in ethanol conversion of 5.16% with an identical catalyst (No. 18 and No. 19). More important, however, is the strong improvement in selectivity to i-butanol by 20% to 97.2%.

The examples 19 to 22 show that the loading of the catalyst has a significant influence on the ethanol conversion. For example, an ethanol conversion of 13.45% can be achieved at 30% loading. However, as a result the very good selectivity of 97.2% (No. 19) is slightly reduced by 4%.

In the embodiment examples 23 to 25, an equivalent catalyst with a Ni:Pt ratio of 95:5 is compared to each of the examples 20 to 22. The highest ethanol conversion is obtained with 10% Ni₉₅Pt₅/C (No. 23). 18.67% ethanol conversion is accompanied by a selectivity of 90.73% to i-butanol. This catalyst thus has a far superior yield to the Pt catalyst due to the higher catalyst stability and activity.

### II.27 Example 27

0.25 g of the catalyst 5% Ni₉₅Pt₅/C (particle size d<75 μm) produced in example 14 is weighed into the autoclave reactor and the autoclave is sealed. The atmosphere is exchanged with nitrogen. 70 mL of the reaction solution is added via a septum. The aqueous reaction solution contains 12.0 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977 GC/MSD mass spectrometer. The results are listed in Table 4.

### II.28 Example 28

0.25 g of the catalyst 5% Ni₉₅Pt₅/C (particle size d<75 μm) produced in example 14 is weighed into the autoclave reactor and the autoclave is sealed. The atmosphere is exchanged with nitrogen. Add 70 mL of the reaction solution via a septum. The methanolic reaction solution contains 12.0 mol/L ethanol (pure, Merck KGaA), 0.45 mol/L NaOH (>97%, Acros Organics) 0.015 mol/L n-decane (99%, Acros Organics) as internal standard. The reaction is carried out at 165° C. for 4 h with a stirrer speed of 1000 rpm and autogenous pressure under nitrogen atmosphere. At 30 min intervals, 3 μL of the reaction solution is quantitatively analyzed by headspace method (full evaporation) on GC-MS (Agilent 8890, 122-7032 UI DB-Wax column) using an Agilent 5977GC/MSD mass spectrometer. The results are listed in Table 4.

TABLE 4

| | | | Selectivity in % | | | |
|---|---|---|---|---|---|---|
| | | X(EtOH) | | | 2-Methyl- | |
| No. | Solvent | % | Acetaldehyde | 1-propanol | 1-propanol | 1-Butanol |
| 27 | Water | 18.56 | 3.24 | — | 0.10 | 23.43 |
| 28 | Methanol | 8.07 | 1.90 | 0.34 | 0.40 | 40.75 |

| | Selectivity in % | | | | | |
|---|---|---|---|---|---|---|
| | 2-Methyl- | 2-ethyl- | 2-Methyl- | | 2-ethyl- | |
| No. | 1-butanol | 1-butanol | 1-pentanol | 1-Hexanol | 1-hexanol | 1-octanol |
| 27 | — | 13.78 | — | 1.44 | 7.31 | 4.15 |
| 28 | 9.41 | 11.12 | 0.14 | 1.64 | 5.46 | 2.91 |

Examples 27 and 28 show the outstanding stability of the catalyst under aqueous conditions. Since no methanol is present in No. 27, no methylation products are formed, whereas these are formed to a small extent in No. 28, since the initial ethanol concentration is decisive for the product selectivity, as already explained. As can be seen from the good conversion of 18%, the catalyst is a water-tolerant catalyst.

Thus, yields of 18.67% i-butanol at 165° C. in 4 h (optimized composition) were obtained with the NiPt catalyst, while the maximum yield of i-butanol with the Pt catalyst reached only 4.68% (at 200° C., 4 h, No. 4).

Overall, the tests show that a stable and economical catalyst is available, which delivers better results than the catalysts from the state of the art. The catalyst shows a lower deactivation rate compared to 5% Pt/C catalysts. In addition, the catalyst is able to generate good conversions even in aqueous solutions. The Pt content is drastically reduced compared to the 5% Pt/C catalyst and the listed catalysts are easy to produce. In addition, a process is proposed which, depending on the initial methanol/ethanol ratio, is able to provide a wide range of products for the chemical industry or a base fuel after simple separation steps. This synthesis is highly tolerant to water, facilitating the purification of the process media.

1. Process for the preparation of C3-C10 mono-alcohols from methanol and ethanol by means of a metal catalyst in the presence of a base, characterized in that, a mixture comprising at least methanol and ethanol is reacted on a carbon-supported metal catalyst comprising at least one alloy of the metals nickel and platinum at a temperature of greater than or equal to 100° C. and less than or equal to 200° C., wherein the molar mass ratio of nickel to platinum, expressed as molar amount of nickel divided by molar amount of platinum, is greater than or equal to 4 and less than or equal to 100.000.

2. The process according to claim 1, wherein the molar mass ratio of nickel to platinum is greater than or equal to 20 and less than or equal to 100.

3. The process according to claim 1, wherein the support material of the carbon-supported metal catalyst comprises greater than or equal to 75% by weight of amorphous carbon.

4. The process according to claim 1, wherein the support material of the carbon-supported metal catalyst has an internal surface area, determined by means of static physisorption of N2, of greater than or equal to 500 m²/g and less than or equal to 1300 m²/g.

5. The process according to claim 1, wherein the temperature is greater than or equal to 130° C. and less than or equal to 170° C.

6. The process according to claim 1, wherein the mass fraction of the metal alloy in the total mass of the catalyst is greater than or equal to 3% by weight and less than or equal to 15% by weight.

7. The process according to claim 1, wherein the carbon-supported metal catalyst has a size distribution with a number-averaged D05 quantile deter-mined via TEM of greater than or equal to 0.5 nm and less than or equal to 5 nm.

8. Metal catalyst at least comprising:
a) a carbonaceous carrier material in a proportion by weight greater than or equal to 80% and less than or equal to 95%;
b) a metal alloy comprising nickel and platinum, wherein the molar proportion of nickel in the alloy is greater than or equal to 80% and less than or equal to 99.99% and the molar pro-portion of platinum in the alloy is greater than or equal to 0.01% and less than or equal to 20%;
wherein the carbon is amorphous.

9. The metal catalyst according to claim 8, wherein the catalyst has an internal surface area, determined by means of static physisorption of N2, of greater than or equal to 500 m2/g and less than or equal to 1300 m2/g.

10. Use of a metal catalyst according to claim 8 for the preparation of C4-C8 mono-alcohols from methanol and ethanol.
